(19) United States
(12) Patent Application Publication
(10) Pub. No.: US 2021/0244539 A1
(43) Pub. Date: Aug. 12, 2021

(54) ANNULOPLASTY RING ASSEMBLY INCLUDING A RING HOLDER WITH A FLEXIBLE SHAFT

(71) Applicant: Edwards Lifesciences Corporation, Irvine, CA (US)

(72) Inventor: Rafael Pintor, Mission Viejo, CA (US)

(21) Appl. No.: 17/244,788

(22) Filed: Apr. 29, 2021

Related U.S. Application Data
(63) Continuation of application No. PCT/US2019/058623, filed on Oct. 29, 2019.
(60) Provisional application No. 62/754,091, filed on Nov. 1, 2018.

(51) Int. Cl. A61F 2/24; A61F 2/95

(52) U.S. Cl. CPC A61F 2/2445; A61F 2220/0075; A61F 2/2466; A61F 2/9517

(57) ABSTRACT

An annuloplasty ring assembly includes an annuloplasty ring configured to be secured to an annulus of a heart valve and a ring holder having a template and a handle attachment assembly. The template includes an upper proximal face, a lower distal face, and a peripheral edge portion configured to receive the annuloplasty ring in conformal contact therewith. The handle attachment assembly includes a flexible shaft and a rigid handle adapter. The flexible shaft has a distal end attached to the template and a proximal end attached to the rigid handle adapter. The flexible shaft is rigid enough to maintain the shape of the annuloplasty ring assembly at a plurality of different positions during implantation while permitting the user to bend the flexible shaft to facilitate access and positioning of the annuloplasty ring on the annulus of the heart valve.

### CROSS-REFERENCE TO RELATED APPLICATION(S)

This application is a continuation of International Patent Application No. PCT/US2019/058623, filed Oct. 29, 2019, which claims the benefit of U.S. Patent Application No. 62/754,091, filed Nov. 1, 2018, the entire contents all of which are incorporated by reference for all purposes.

This application is related to U.S. Patent Application No. 62/754,066, filed Nov. 1, 2018, and to U.S. Patent Application No. 62/754,070, filed Nov. 1, 2018, the entire disclosures of both applications incorporated by reference herein for all purposes.

### TECHNICAL FIELD

The present disclosure relates generally to medical devices and to tools for delivering such medical devices. More specifically, the disclosure relates to surgical repair of heart valves that have malformations and/or dysfunctions. Embodiments of the invention relate to holders and methods for facilitating repair of heart valves with annuloplasty rings, e.g., mitral or tricuspid annuloplasty rings.

### BACKGROUND

Referring to FIG. 1, the human heart is generally separated into four pumping chambers which pump blood through the body. Each chamber is provided with its own one-way exit valve. The left atrium receives oxygenated blood from the lungs and advances the oxygenated blood to the left ventricle through the mitral (or bicuspid) valve. The left ventricle collects the oxygenated blood from the left atrium and pushes it through the aortic valve to the aorta, where the oxygenated blood is then distributed to the rest of the body. Deoxygenated blood from the body is then collected at the right atrium and advanced to the right ventricle through the tricuspid valve. The right ventricle then advances the deoxygenated blood through the pulmonary valve and the pulmonary artery to the lungs to again supply the blood with oxygen.

Each of the valves associated with the chambers of the heart are one-way valves that have leaflets to control the directional flow of the blood through the heart, and to prevent backflow of the blood into other chambers or blood vessels that are upstream of the particular chamber. The valves are each supported by an annulus having a dense fibrous ring attached either directly or indirectly to the atrial or ventricular muscle fibers.

When a valve becomes diseased or damaged, the efficiency and/or general functionality of the heart may be compromised. Diseased heart valves may be categorized as either stenotic, wherein the valve does not open sufficiently to allow adequate forward flow of blood through the valve, and/or incompetent, wherein the valve does not close completely, causing excessive backward flow of blood through the valve when the valve is closed. Valve disease can be severely debilitating and even fatal if left untreated.

Various surgical techniques may be used to repair a diseased or damaged valve. One method for treating defective valves is through repair or reconstruction. One repair technique that has been shown to be effective in treating incompetence is annuloplasty, in which the effective size and/or shape of the valve annulus is modified by securing a repair segment, such as an annuloplasty ring, around all or a portion of the heart valve annulus. For example, the valve annulus may be contracted by attaching a prosthetic annuloplasty ring to an interior wall of the heart around the valve annulus. The annuloplasty ring is designed to support the functional changes that occur during the cardiac cycle, maintaining coaptation and valve integrity to prevent reverse flow while permitting good hemodynamics during forward flow.

The annuloplasty ring typically comprises an inner substrate, often formed from a metal (such as stainless steel or titanium) or from a flexible material (such as silicone rubber or Dacron cordage), which is typically covered with a biocompatible fabric or cloth, forming a sewing cuff, to allow the ring to be sutured to the heart tissue. Annuloplasty rings include annuloplasty bands and may be stiff or flexible, may be split (including rings that extend at least halfway around the valve annulus) or continuous, and may have a variety of shapes, including circular, D-shaped, C-shaped, saddle-shaped, and/or kidney-shaped. Examples are seen in U.S. Pat. Nos. 5,041,130, 5,104,407, 5,201,880, 5,258,021, 5,607,471, 6,187,040, and 6,805,710, the contents of which are incorporated herein by reference in their entirety. Many annuloplasty rings are formed in a plane, but some rings are generally non-planar. Such non-planar rings can be saddle-shaped, and/or bowed along various portions, such as being bowed along their anterior or straight side to conform to the desired shape of the annulus at that location.

One of the most frequently used annuloplasty rings is the partially flexible Carpentier-Edwards Physio® ring available from Edwards Lifesciences of Irvine, Calif. The Physio ring is a “semi-rigid” ring because it offers selective flexibility at the posterior section while preserving the remodeling effect through a rigid anterior section.

In the usual mitral annuloplasty ring implant procedure, an array of separate implant sutures are first looped through all or portions of the exposed mitral annulus at intervals spaced equidistant from one another, such as, for example, about 4 mm intervals. The surgeon then threads the implant sutures through the annuloplasty ring at more closely spaced intervals, such as, for example, about 2 mm. This occurs with the prosthesis outside the body, typically secured to a peripheral edge portion of a holder or template. The ring on the holder is then advanced (parachuted) distally along the array of pre-anchored implant sutures into contact with the valve annulus, thus effecting a reduction in valve annulus circumference. At this point a handle used to manipulate the holder or template may be detached for greater visibility of the surgical field. The surgeon ties off the implant sutures on the proximal side of the ring, and releases the ring from the holder or template, typically by severing connecting sutures at a series of cutting guides. Examples of annuloplasty ring implant procedures are described in U.S. Pat. Nos. 8,216,304 and 8,152,844, the contents of which are incorporated herein by reference in their entirety.

Implanting an annuloplasty ring using minimally invasive procedures can be difficult. Using a standard atriotomy approach to the mitral valve for repair, the surgeon does not generally achieve straight-on access to the mitral valve annulus. Instead, the surgeon often sees and accesses the valve from an angle. Therefore, when the sutures are placed in the annuloplasty ring and it is “parachuted” down to the annulus, the ring and holder must be reoriented to sit flat against the annulus. In minimally-invasive (MIS) approaches to the mitral valve, the annuloplasty ring must be passed through a relatively small port into the chest. This can be complicated by the bulk of some holders, so in some instances a surgeon simply removes the holder from the ring prior to “parachuting” the ring down the sutures and onto the valve annulus. However, the removal of the entire holder from the ring can make control of the annuloplasty ring more difficult.

Many surgeons prefer to keep the holder attached to the ring during the initial procedure of accessing the valve annulus. The existing design of the holder for the Carpentier-Edwards Physio ring, however, includes a rigid arm or shaft that extends proximally more than about 4 cm (about 1.5 inches). A handle, attached to the rigid arm, has a rigid distal end, such that the combined length of the holder at the rigid arm and the rigid distal end of the handle is greater than about 5 cm (about 2 inches), e.g., about 5.5 cm (about 2¼″) long from the annuloplasty ring and the plane of the valve annulus. This long rigid length of the assembly is undesirable because it prevents the surgeon from bending the shaft and adjusting the access angle of the annuloplasty ring near the plane of the valve annulus during deployment of the ring. This adjustment of the ring access angle is desirable particularly in minimally invasive procedures where access and operating space are limited and the plane of the ring may not align well to the plane of the valve annulus. A better access angle would result in better alignment of the ring's plane with respect to the plane of the valve annulus.

Despite numerous designs presently available or proposed in the past, there is a need for an improved holder for annuloplasty rings and prosthetic valves that provides a flexible shaft near the plane of the ring or valve and that allows the device and angle to be shaped as needed to achieve the best possible access angle to match that of the valve annulus during deployment. The holder described below eliminates this problem by introducing a flexible segment in the previously rigid section of the shaft at a location adjacent the ring thereby enabling the surgeon to achieve excellent access every time.

### SUMMARY

In a preferred embodiment of the present invention, an annuloplasty ring assembly includes an annuloplasty ring configured to be secured to an annulus of a heart valve and a ring holder comprising a template and a handle attachment assembly. The template includes an upper proximal face, a lower distal face, and a peripheral edge portion configured to receive the annuloplasty ring in conformal contact therewith. The handle attachment assembly includes a flexible shaft and a rigid handle adapter. The flexible shaft has a distal end attached to the template and a proximal end attached to the rigid handle adapter. The flexible shaft is rigid enough to maintain the shape of the annuloplasty ring assembly at a plurality of different positions during implantation while permitting the user to plastically deform or bend the flexible shaft to facilitate access and positioning of the annuloplasty ring on the annulus of the heart valve.

In a preferred embodiment, the flexible shaft has a flexing portion within about 4 cm (about 1.5 inches) of the upper proximal face of the template. In another embodiment, the flexible shaft has a flexing portion located at a distance to the upper proximal face of the template that is shorter than a length measured across the template from one edge to an opposite edge along a minor axis.

In an alternative embodiment, the template includes a rigid grip projecting up from the upper proximal face with the distal end of the flexible shaft attached to the rigid grip. The rigid grip of the template is configured to be gripped by the gripping feature of a tool.

In another embodiment, the annuloplasty ring assembly described above includes a handle. In addition, the rigid handle adapter has a distal end that is attached to the proximal end of the flexible shaft and a proximal end has an attachment feature that is releasably securable to an attachment feature of the handle. In a preferred embodiment, the handle also has a flexible shaft that is rigid enough to maintain the shape of the annuloplasty ring assembly at a plurality of different positions during implantation while permitting the user to plastically deform or bend the flexible shaft of the handle to facilitate access and positioning of the annuloplasty ring on the annulus of the heart valve.

In another embodiment, an annuloplasty ring assembly includes a handle and a ring holder. The ring holder includes a template and a handle attachment assembly. The template has an upper proximal face, a lower distal face, and a peripheral edge portion configured to receive an annuloplasty ring in conformal contact therewith. The handle attachment assembly includes a flexible shaft and a rigid handle adapter. The flexible shaft has a distal end attached to the template and a proximal end attached to the rigid handle adapter. The rigid handle adapter is releasably securable to the handle and the flexible shaft is rigid enough to maintain the shape of the annuloplasty ring assembly at a plurality of different positions during implantation while permitting the user to plastically deform or bend the flexible shaft to facilitate access and positioning of the annuloplasty ring on the annulus of the heart valve. Preferably, the template further comprises a rigid grip projecting up from the upper proximal face and the distal end of the flexible shaft is attached to the rigid grip.

In a preferred embodiment, the flexible shaft has a flexing portion within about 4 cm (about 1.5 inches) of the upper proximal face of the template and/or the template has a major axis and a minor axis and the flexible shaft has a flexing portion located at a distance to the upper proximal face of the template that is shorter than a length measured across the template from one edge to an opposite edge along the minor axis.

Furthermore, the handle preferably has a flexible shaft that is rigid enough to maintain the shape of the annuloplasty ring assembly at a plurality of different positions during implantation while permitting the user to plastically deform or bend the flexible shaft of the handle to facilitate access and positioning of the annuloplasty ring on the annulus of the heart valve.

Other embodiments provide a method for delivering and implanting an annuloplasty ring in a patient in need thereof using a holder or annuloplasty ring assembly disclosed herein.

### BRIEF DESCRIPTION OF THE DRAWINGS

Further features and advantages of the invention will become apparent from the description of embodiments using the accompanying drawings. In the drawings:

FIG. 1 is a schematic cross-sectional view of a human heart;

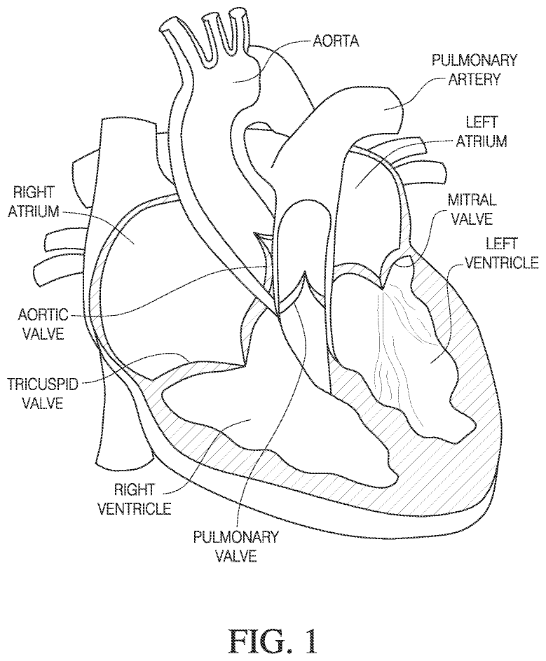

FIG. 2 is a perspective view of an assembled annuloplasty ring and ring holder and a detachable handle according to an embodiment of the invention;

FIG. 3 is an exploded perspective view of the annuloplasty ring and a template of the ring holder of FIG. 2;

FIG. 4 is an assembled perspective view of the ring holder of FIG. 2 without the annuloplasty ring;

FIG. 5 is an exploded view of the ring holder of FIG. 4;

FIG. 6 is a bottom perspective view of the template of the ring holder of FIG. 4;

FIG. 7 is a front view from the posterior side of the template of the ring holder of FIG. 4; and

FIG. 8 is a side view of the template of the ring holder of FIG. 4.

### DETAILED DESCRIPTION

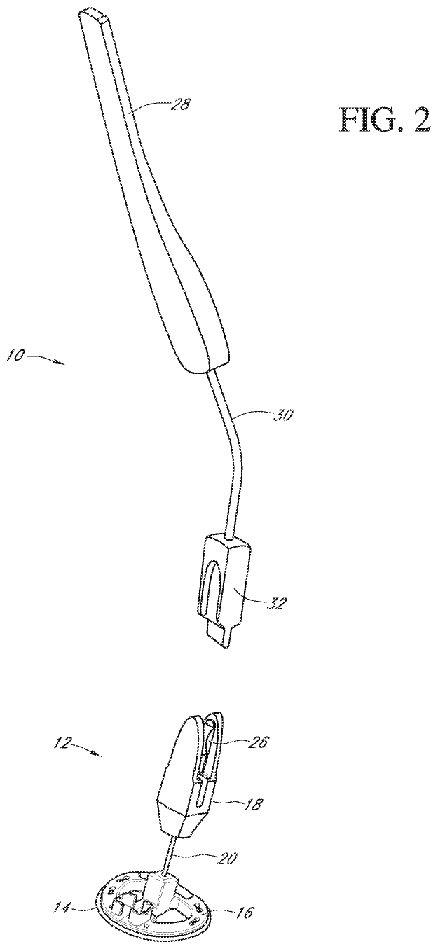

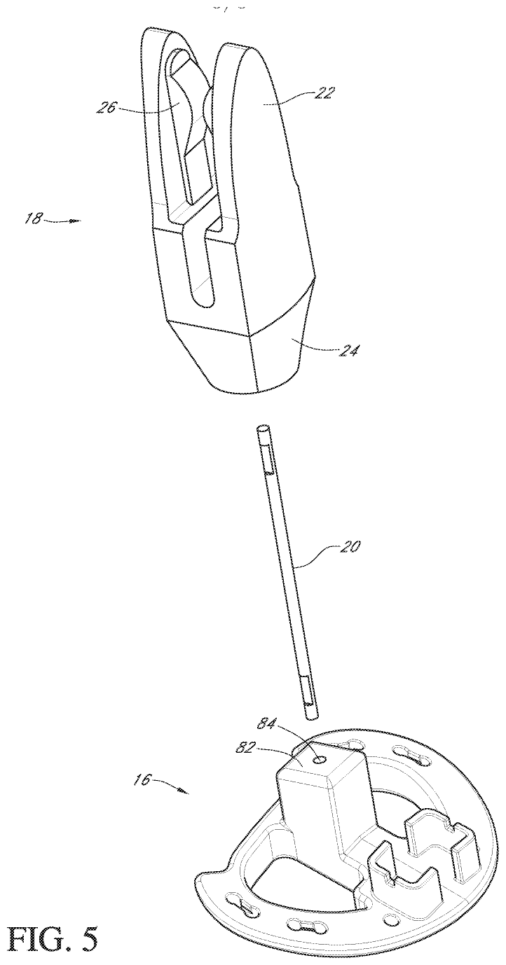

With reference to FIGS. 2-5, one embodiment of the annuloplasty ring assembly of the present invention includes a handle 10, a ring holder 12, and an annuloplasty ring 14. The ring holder 12 has a lower rigid template 16 secured to an upper rigid handle adapter 18 by a flexible shaft 20, such as a malleable or plastically deformable shaft.

The handle adapter 18 has a proximal end 22 and a distal end 24, with the distal end 24 secured to the flexible shaft 20. The handle adapter proximal end 22 includes an attachment feature 26 by which the handle 10 can be removably secured to the handle adapter 18.

The handle 10 includes a rigid proximal portion 28, a middle portion 30, and a rigid distal portion 32. The distal portion 32 has an attachment feature that is configured to cooperate with the attachment feature 26 of the handle adapter 18 in order to secure the handle 10 to the handle adapter 18. The middle portion 30 of the handle may be rigid or it may have flexible properties, such as malleability, thereby permitting a surgeon to adjust (via bending) the handle to a configuration convenient for the particular application and surgeon.

Attachment and removal of the handle 10 to and from the handle adapter 18 is permitted by a snap fit (not shown). The handle 10 can be secured to the handle adapter 18 by snapping the attachment feature of the distal portion 32 of the handle into the attachment feature 26. Removing the handle 10 is accomplished by gripping the ring holder 12 while pulling the handle 10.

The ring holder 12 includes multiple sections of variable rigidity. In particular, the flexible shaft 20 is located adjacent to the annuloplasty ring. This flexible shaft provides the user with additional degrees of freedom to more easily and readily position the handle to be able to easily align the plane of the annuloplasty ring with respect to the plane of the valve annulus, for example, by plastically deforming the flexible shaft as desired.

Accordingly, the ring holder 12 is composed of three basic sections:

(a) the lower rigid template 16 where the ring holder 12 is attached to the annuloplasty ring 14;

(b) the middle flexible shaft 20 that joins the rigid template 16 and the handle adapter 18 of the ring holder 12; and,

(c) the upper rigid handle adapter 18 where the ring holder 12 connects to the handle 10.

The middle flexible shaft 20 is a shapeable section that may be made of multiple materials such as Nitinol, stainless steel, flexible polymer, or any material with the desired mechanical flexible properties such as malleability. To manufacture this design, the rigid template 16 and rigid handle adapter 18 may be separate monolithic polymer based sections that can be over molded over each end of the flexible shaft 20. Alternative assembly methods include adhesive bonding, press fitting, the use of threaded connections or a combination of all these assembly methods.

In this embodiment, the flexible shaft 20 is rigid enough to maintain the shape of the assembly at a plurality of different positions during the implantation procedure while also providing the user with the ability to bend and/or reposition the flexible shaft as necessary to facilitate access and positioning of the annuloplasty ring on the valve annulus.

Ring holders of the present invention may accommodate annuloplasty rings that are open or discontinuous (e.g., C-shaped) or closed or continuous (e.g., D-shaped). The rings may be rigid, flexible, or semi-flexible. The ring holders of the present invention may conform to planar or nonplanar rings, and are adaptable to rings used to repair any of the valves within the heart. Further, the holders of the present invention may be utilized to hold heart valves.

The holders of the present invention are especially suitable for annuloplasty rings, including annuloplasty bands, that are generally rigid and will resist distortion when subjected to the stress imparted thereon by the mitral valve annulus of an operating human heart. In this sense, “distortion” means substantial permanent deformation from a predetermined or manufactured shape. A number of generally rigid materials can be utilized as an inner core of the rings that will perform this function, including various bio-compatible polymers and metals and/or alloys. Certain polyesters that resist distortion and also rapid degradation within the body may be used. In a preferred embodiment, at least an inner core or body of the annuloplasty ring of the present invention is made of a suitable metal, such as ELGILOY® made by Elgiloy, L.P. of Elgin, Ill., U.S.A, or also titanium or its alloys. The core or ring body may be one piece, or may include a plurality of concentric or otherwise cooperating elements.

Furthermore, the annuloplasty ring holders of the present invention are also especially suited to hold annuloplasty rings designed to correct particular pathologies. That is, holders may be provided for a set of rings defined by ring bodies wherein the proportional shapes of the ring bodies change with increasing nominal orifice sizes of the ring bodies in the set. The change of ring shape depends on the pathology being corrected. For instance, pathologies resulting in mitral regurgitation may benefit from a set of rings which have increasing circularity as the ring size increases.

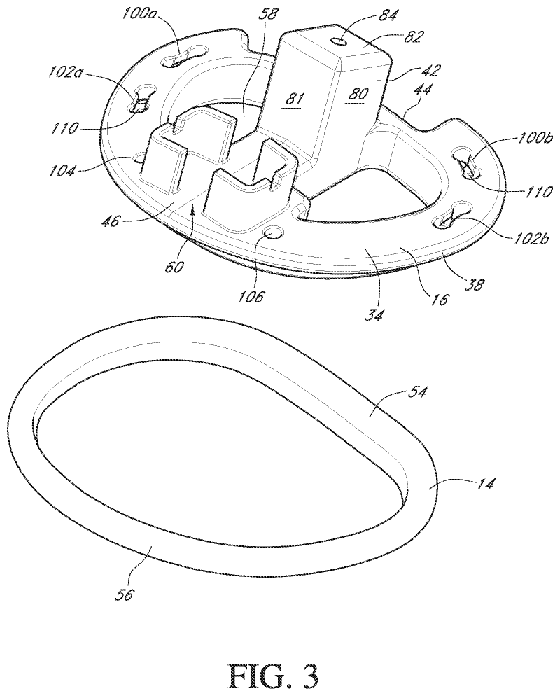

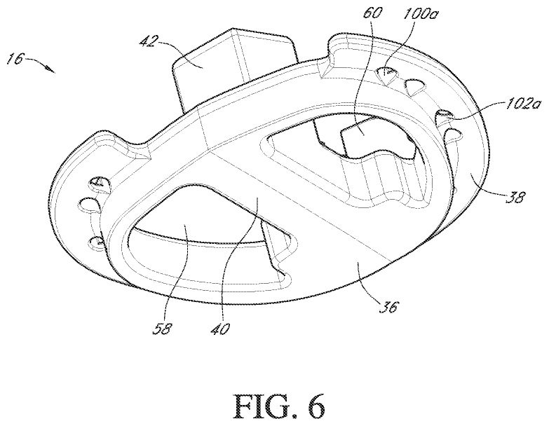

With reference to FIGS. 3 and 6-8, the rigid template 16 is defined by an upper proximal face 34, a lower distal face 36, an outer peripheral edge portion 38 and a crossbar 40 extending from one side of the peripheral edge portion to another. The peripheral edge portion 38 has the same shape as the annuloplasty ring 14 that it is designed to hold, and thus is somewhat D-shaped and defines a major axis dimension D₁, (across the crossbar 40) and a minor axis dimension D₂ (along the crossbar 40). The crossbar extends along the minor axis dimension of the template 16, with a rigid grip 42 located adjacent the peripheral edge portion 38 at an anterior segment 44 of the template 16. Between the peripheral edge portion 38 and the crossbar 40, template 16 provides a pair of relatively large visibility windows 58 that together occupy a large cross-sectional area within the peripheral edge portion. The windows 58 allow the surgeon to see distally through the template 16 and the annuloplasty ring 14 to evaluate the condition of the mitral annulus as the ring is implanted. In a similar manner, if the template 16 is utilized for holding a prosthetic valve, the windows 58 provide enhanced visibility of the prosthetic valve leaflet structure.

The rigid grip 42 projects upwardly (or proximally) from the upper proximal face 34 of the template 16. Preferably, the grip 42 has a cube shape with first and second pairs of parallel side walls 80, 81 to permit gripping the template with a tool. A top face 82 of the grip has an opening 84 to receive the distal end of the flexible shaft 20. The height of the grip 42 is sufficient to permit gripping by a tool.

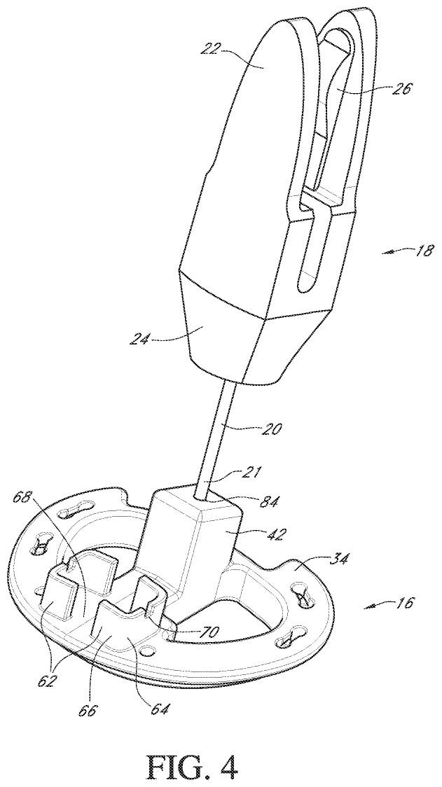

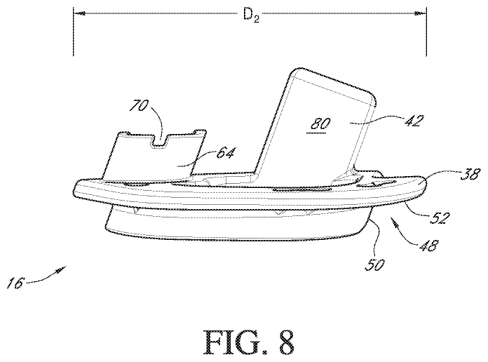

With reference to FIG. 4, the flexible shaft 20 has a flexing portion 21 adjacent the opening 84 of the grip 42 that is within about 4 cm (about 1.5 inches) of the upper proximal face 34 of the template (e.g., at the base of the grip). Alternatively, the flexing portion is within about 4 cm (about 1.5 inches) of a point located in a center space of the annuloplasty ring and intersected by a line along an axis of the opening 84. In a further embodiment, the flexing portion 21 of the flexible shaft 20 is closer to the upper proximal face 34 of the template than a length measured across the template from an outermost point of the edge portion 38 to an opposite outermost point of the edge portion along the minor axis D₂ (see FIG. 8). Such placement of the flexing portion 21 of the flexible shaft 20 near the upper proximal face 34 of the template 16 allows the ring holder 12 to be bent as needed to achieve a good access angle for deploying the annuloplasty ring as mentioned above. The ability to bend and shape the ring holder 12 as needed also enables the surgeon to maximize visibility of the work space by shaping the handle out of the field of view.

In other embodiments, a projecting grip 42 is not provided, and the distal end of the flexible shaft 20 directly enters the upper proximal face 34 of the template with the flexing portion 21 directly adjacent thereto at a location that allows bending.

Notably, the location of the flexible shaft 20 of the ring holder 12 adjacent the upper proximal face 34 of the template 16 provides several advantages during the implantation procedure. A surgeon or a member of the surgical staff can bend the ring holder's malleable shaft 20 at any stage of the annuloplasty ring implantation procedure, from the beginning when the surgeon is placing sutures through the sewing cuff of the implant up to the point where the surgeon is ready to parachute the ring onto the valve annulus. Bending the shaft when the sutures are being placed through the sewing cuff can be done to facilitate suture placement by providing a more appropriate angle of the implant for the surgeon to work with or for the assistant to hold the implant/handle. At another step of the procedure, when the surgeon is ready to parachute the ring into place and depending on the access (e.g. full sternotomy, lower mini-sternotomy or right lateral thoracotomy) and plane of the mitral valve, the surgeon may bend the flexible shaft 20 to adjust the plane of the implant to facilitate access and better match the plane of the valve annulus for easier and better deployment. Alternatively, or in addition, the malleable or flexible middle portion 30 of the handle 10 may be bent as desired.

In a minimally invasive setting where the size of the incision is very small such as a right lateral thoracotomy approach, the surgeon can adjust the shape of the ring/holder assembly to facilitate insertion. Once the ring holder assembly goes through the small incision, the flexible shaft 20 can be bent back to the desired approach angle using a right handle hemostat (not shown). The rigid grip 42 of the ring holder 12 provides the grab point for the hemostasis or any other tool used.

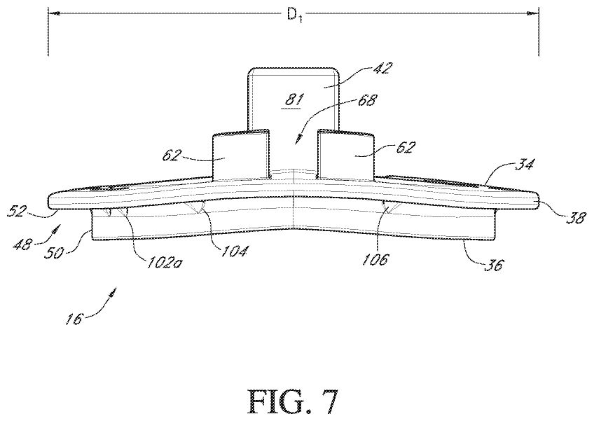

With reference now to FIG. 7, an annuloplasty ring (not shown) will conform to an angled channel 48 defined by a generally axially extending distal wall 50 and an outwardly extending proximal ledge 52 forming an outer extent of the template of the peripheral edge portion 38. The annuloplasty ring will extend inside and around the channel and follow a three-dimensional path and the peripheral edge portion 38 and the channel 48 mirror this three-dimensional shape.

With reference again to FIG. 3, the annuloplasty ring 14 is designed for repair of the mitral annulus, and includes an anterior segment 54 opposite a posterior segment 56. Likewise, the template 16 defines the anterior segment 44 and a posterior segment 46. A single cutting well 60 projects upward from the proximal face 34 on the template 16, adjacent the posterior segment 46. The cutting well 60 is diametrically opposed across the template 16 from the grip 42 and adjacent the peripheral edge portion 38.

The distance between the cutting well 60 adjacent the posterior segment 46 and the grip 42 provides ample space for a surgeon to manipulate a cutting instrument within the surgical field. Moreover, the single cutting well 60 presents the only length of a suture or filament connecting the annuloplasty ring 14 to the template 16 that extends above the proximal face 34. This combination of features provides a one cut release structure that is highly visible to the surgeon. The filament suspended across the cutting well 60 is essentially the only length of filament visible looking down on the proximal face 34.

The proximal face 34 of the template 16 desirably comprises a substantially smooth upper surface that extends at least around the peripheral edge portion 38. Certain features of the present invention are defined relative to the proximal face 34. For instance, some features are recessed below the proximal face, emerge above it, or are visible on the proximal face. The single cutting well 60 projects relatively high above the proximal face 34 and is located near the peripheral edge portion 38 and opposite from the grip 42 so as to be highly visible. Certain other features, as will be explained below, may be exposed to the proximal side of the template but are relatively small and/or recessed in the proximal face 34 so as to be much less apparent, especially in the usually bloody environment of the surgical field.

With reference to FIG. 4, walls 62 of the cutting well 60 are located adjacent the peripheral edge portion 38 and extend upward from the proximal face 34. Each wall 62 includes a relatively straight section 64 parallel to the other wall, and opposite ends 66 that curve inward toward the other wall. The inwardly curved ends form a gap 68 between the two walls 62 to help guide a cutting implement such as a scalpel (not shown) into a midplane between the two walls. A notch 70 is formed on an upper edge of each wall 62. The combination of the notches 70 across the two walls 62 provides a convenient bridge across which connecting suture filaments are suspended. The walls 62 present one configuration of cutting well that may be utilized, and of course others are contemplated. Cutting wells desirably project upward from the proximal face 34 of the template 16 so that a connecting filament can be suspended over a space within which a cutting implement can be inserted. Alternatively, a cutting well that is recessed below the proximal face may also be used, although the visibility and accessibility are somewhat reduced.

With reference to FIGS. 3 and 7, the template 16 includes a series of through holes for passage of a connecting filament (not shown) for firmly holding the annuloplasty ring 14 in the angled channel 48. It should be understood that although through holes are the preferred construction, other configurations that provide passages through the template 16 and/or perform similar functions are contemplated. For example, the template 16 is provided with a pair of cleats 100a, 100b adjacent the peripheral edge portion 38 and on the anterior segment 44. In the illustrated embodiment, each cleat comprises a pair of closely-spaced holes that pass entirely through the proximal ledge 52, such that the angled channel 48 communicates with the space above the proximal face 34. A short bridge portion 110 recessed from the proximal face 34 connects the two holes. A flexible connecting filament may be looped through the two holes and tied to itself so as to anchor the filament to the cleat 100. As will be apparent to one of skill in the art, forming or machining a pair of through holes through the proximal ledge 52 is relatively economical, and in addition the process of assembling the annuloplasty ring 14 to the template 16 using these through holes is relatively simple. However, a cleat 100 that has only a single through hole or does not utilize through holes at all is entirely within the scope of the present invention. For instance, a free end of the filament may be secured to a small projection or hook provided on the template 16, rather than looping the end through the two through holes.

The cleats 100a, 100b are spaced apart around the peripheral edge portion 38, preferably equidistantly from the cutting well 60. As the cutting well 60 is located adjacent the peripheral edge portion 38 on the posterior segment 46, the cleats 100a, 100b, being located on the anterior segment 44, are circumferentially spaced by at least 90 degrees around the template 16 from the cutting well 60. A primary flexible connecting filament (not shown) has free ends anchored to the cleats 100a, 100b and a midportion that passes around the posterior segment 46 of the template 16 and over the cutting well 60.

The template 16 also includes a pair of filament loops 102a, 102b, each spaced between a corresponding cleat 100 and the cutting well 60. Again, each loop 102a, 102b comprises a pair of closely-spaced holes that pass entirely through the proximal ledge 52, such that the angled channel 48 communicates with the space above the proximal face 34. A short bridge portion 110 recessed from the proximal face connects the two holes. A flexible connecting filament may be looped through the two holes to pass from within channel 48 over the recessed bridge 110 and back into the channel 48.

The template 16 also includes a pair of cutting well apertures 104, 106 spaced on either side of the cutting well 60. As with the cleats 100a, 100b and loops 102a, 102b the apertures 104, 106 desirably pass entirely through the proximal ledge 52, such that the angled channel 48 communicates with the space above the proximal face 34.

It will be appreciated that routing sutures between the annuloplasty ring 14 and the ring holder 12 should be attached to the template to insure that those sutures are removed together with the ring holder after the annuloplasty ring is delivered to the native valve annulus. Examples of suture routing are described in U.S. Pat. No. 8,152,844, the contents of which are incorporated herein by reference in their entirety.

To remove the ring 14 from the holder 12, the user will cut the retaining sutures that hold the ring 14 to the holder 12. A scalpel or other cutting instrument is passed into the gap 68 of the cutting well 60 to cut the retaining sutures. With the retaining sutures cut, the holder 12 can be pulled away from the ring 14. Note that in an exemplary embodiment the retaining sutures are connected via knots or other restraints to the holder 12, so that when the retaining sutures are cut their severed ends will remain connected to the holder 12. Accordingly, when the retaining sutures are cut and the holder 12 pulled away from the ring 14, the retaining sutures will be removed from the ring 14 along with the holder 12.

For purposes of this description, certain aspects, advantages, and novel features of the embodiments of this disclosure are described herein. The disclosed methods, apparatus, and systems should not be construed as being limiting in any way. Instead, the present disclosure is directed toward all features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms can vary depending on the particular implementation and are readily discernible by one of ordinary skill in the art.

In view of the many possible embodiments to which the principles of the disclosure can be applied, it should be recognized that the illustrated embodiments are only preferred examples and should not be taken as limiting the scope of the disclosure. Rather, the scope of the disclosure is defined by the following claims.

What is claimed is:

1. An annuloplasty ring assembly, comprising:
an annuloplasty ring configured to be secured to an annulus of a heart valve; and
a ring holder comprising a template and a handle attachment assembly, the template comprising an upper proximal face, a lower distal face, and a peripheral edge portion configured to receive the annuloplasty ring in conformal contact therewith;
wherein the handle attachment assembly comprises a flexible shaft and a rigid handle adapter, the flexible shaft having a distal end attached to the template and a proximal end attached to the rigid handle adapter, the rigid handle adapter having an attachment feature that cooperates with an attachment feature of a handle; and
wherein the flexible shaft is rigid enough to maintain the shape of the annuloplasty ring assembly at a plurality of different positions during implantation while permitting the user to plastically deform the flexible shaft to facilitate access and positioning of the annuloplasty ring on the annulus of the heart valve.

2. The annuloplasty ring assembly of claim 1, wherein the template further comprises a rigid grip projecting up from the upper proximal face and the distal end of the flexible shaft is attached to the rigid grip.

3. The annuloplasty ring assembly of claim 1, wherein the annuloplasty ring is secured to the peripheral edge portion of the template.

4. The annuloplasty ring assembly of claim 3, wherein the annuloplasty ring is secured to the peripheral edge portion of the template by sutures.

5. The annuloplasty ring assembly of claim 1, further comprising a handle, and wherein the rigid handle adapter has a distal end that is attached to the proximal end of the flexible shaft and a proximal end that has the attachment feature that cooperates with and is releasably securable to an attachment feature of the handle.

6. The annuloplasty ring assembly of claim 5, wherein the handle is releasably secured to the proximal end of the rigid handle adapter.

7. The annuloplasty ring assembly of claim 2, further comprising a tool having a gripping feature, and wherein the rigid grip of the template is configured to be gripped by the gripping feature of the tool.

8. The annuloplasty ring assembly of claim 7, wherein the rigid grip comprises first parallel side walls of the rigid grip that are configured to be gripped by the gripping feature of the tool.

9. The annuloplasty ring assembly of claim 8, wherein the rigid grip comprises second parallel side walls of the rigid grip that are configured to be gripped by the gripping feature of the tool, the second parallel side walls located between the first parallel side walls.

10. The annuloplasty ring assembly of claim 1, wherein the flexible shaft has a flexing portion within about 4 cm (about 1.5 inches) of the upper proximal face of the template.

11. The annuloplasty ring assembly of claim 1, wherein the template has a major axis and a minor axis and wherein the flexible shaft has a flexing portion located at a distance to the upper proximal face of the template that is shorter than a length measured across the template from one edge to an opposite edge along the minor axis.

12. The annuloplasty ring assembly of claim 1, further comprising a handle that is releasably secured to the handle adapter.

13. The annuloplasty ring assembly of claim 12, wherein the handle has a flexible shaft that is rigid enough to maintain the shape of the annuloplasty ring assembly at a plurality of different positions during implantation while permitting the user to plastically deform the flexible shaft of the handle to facilitate access and positioning of the annuloplasty ring on the annulus of the heart valve.

14. An annuloplasty ring assembly, comprising:
a handle; and
a ring holder comprising a template and a handle attachment assembly, the template comprising an upper proximal face, a lower distal face, and a peripheral edge portion configured to receive an annuloplasty ring in conformal contact therewith;
wherein the handle attachment assembly comprises a flexible shaft and a rigid handle adapter, the flexible shaft having a distal end attached to the template and a proximal end attached to the rigid handle adapter;
wherein the rigid handle adapter is releasably securable to the handle; and
wherein the flexible shaft is rigid enough to maintain the shape of the annuloplasty ring assembly at a plurality of different positions during implantation while permitting the user to plastically deform the flexible shaft to facilitate access and positioning of the annuloplasty ring on the annulus of the heart valve.

15. The annuloplasty ring assembly of claim 14, wherein the template further comprises a rigid grip projecting up from the upper proximal face and the distal end of the flexible shaft is attached to the rigid grip.

16. The annuloplasty ring assembly of claim 15, further comprising a tool having a gripping feature, and wherein the rigid grip of the template comprises first parallel side walls of the rigid grip that are configured to be gripped by the gripping feature of the tool.

17. The annuloplasty ring assembly of claim 14, wherein the rigid handle adapter has a distal end that is attached to the proximal end of the flexible shaft and a proximal end that has the attachment feature that cooperates with and is releasably secured to an attachment feature of the handle.

18. The annuloplasty ring assembly of any of claim 14, wherein the flexible shaft has a flexing portion within 1.5 inches of the upper proximal face of the template.

19. The annuloplasty ring assembly of any of claim 14, wherein the template has a major axis and a minor axis and wherein the flexible shaft has a flexing portion located at a distance to the upper proximal face of the template that is shorter than a length measured across the template from one edge to an opposite edge along the minor axis.

20. The annuloplasty ring assembly of any of claim 14, wherein the handle has a flexible shaft that is rigid enough to maintain the shape of the annuloplasty ring assembly at a plurality of different positions during implantation while permitting the user to plastically deform the flexible shaft of the handle to facilitate access and positioning of the annuloplasty ring on the annulus of the heart valve.
